(19) United States
(12) Patent Application Publication
(10) Pub. No.: US 2010/0130408 A1
(43) Pub. Date: May 27, 2010

(54) USE OF MODIFIED CYCLOSPORINS

(76) Inventors: Motoyuki Kohjima, Fukouka (JP); Makoto Nakamuta, Fukuoka (JP)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/444,941

(22) PCT Filed: Oct. 10, 2007
(86) PCT No.: PCT/EP2007/060794

§ 371 Date: Dec. 1, 2009

Related U.S. Application Data
(60) Provisional application No. 60/829,228, filed on Oct. 12, 2006.

(51) Int. Cl. A61K 38/13; A61P 37/06; A61P 1/16; C07K 7/64; C12N 5/02

(52) U.S. Cl. 514/11; 530/317; 435/375

(57) ABSTRACT

Disclosed are non-immunosuppressive cyclophilin-binding cyclosporine having useful properties in the prevention or treatment of liver diseases.

The present invention relates to a new use for non-immunosuppressive cyclosporins.

The cyclosporins comprise a class of structurally distinctive, cyclic, poly-N-methylated undecapeptides, commonly possessing pharmacological, in particular immuno-suppressive, or anti-inflammatory activity. Cyclosporins which bind strongly to cyclophilin but are not immunosuppressive have been identified. WO2005021028 A1 discloses the use of non-immunosuppressive cyclosporins have an inhibitory effect on Hepatitis C virus (HCV).

A cyclosporin is considered to be non-immunosuppressive when it has an activity in the Mixed Lymphocyte Reaction (MLR) of no more than 5%, preferably no more than 2%, that of cyclosporin A. The Mixed Lymphocyte Reaction is described by T. Meo in “Immunological Methods”, L. Lefkovits and B. Peris, Eds., Academic Press, N.Y. pp. 227-239 (1979). Spleen cells (0.5×106) from Balb/c mice (female, 8-10 weeks) are co-incubated for 5 days with 0.5×105 irradiated (2000 reds) or mitomycin C treated spleen cells from CBA mice (female, 8-10 weeks). The irradiated allogeneic cells induce a proliferative response in the Balb/c spleen cells which can be measured by labeled precursor incorporation into the DNA. Since the stimulator cells are irradiated (or mitomycin C treated) they do not respond to the Balb/c cells with proliferation but do retain their antigenicity. The IC₅₀ found for the test compound in the MLR is compared with that found for cyclosporin A in a parallel experiment. In addition, non-immunosuppressive cyclosporins lack the capacity of inhibiting CN and the downstream NF-AT pathway.

Fibrosis is the formation or development of excess fibrous connective tissue in an organ or tissue as a reparative or reactive process. One form of fibrosis, cirrhosis, is a consequence of chronic liver disease characterized by replacement of liver tissue by fibrotic scar tissue as well as regenerative nodules, leading to progressive loss of liver function. Hepatic stellate cells (HSCs) are monparenchymal liver cells which have a characteristic stellate morphology and reside in the perisinusoidal space of Disse. Following liver injury, HSCs undergo transdifferentiation to an activated myofibroblastic phenotype and express of ∝-smooth muscle actin. Activated HSCs then proliferate and produce extracellular matrix proteins such as collagens. Previous evaluation of the effects of immunosuppressive drugs, such as cyclosporine and tacrolimus, on cell proliferation and collagen production in HSCs, has found that cyclosporine suppressed cell growth and collagen production but tacrolimus did not have such effects, indicating that cyclosporine potentially have an anti-fibrogenic effect.

Currently available antifibrotic therapies have been directed against suppressing hepatic inflammation rather than subduing fibrosis. Points of therapeutic intervention are needed that include efforts to remove the injurious stimuli, suppress hepatic inflammation, downregulate stellate cell activation, and promote matrix degradation.

Accordingly, the present invention provides the use of a non-immunosuppressive cyclophilin-binding cyclosporin in the prevention or treatment of liver diseases such as graft-cirrhosis, chronic hepatitis, cirrhosis, liver cancer, e.g. hepatocellular carcinoma or the progression thereof. Further, the non-immunosuppressive cyclophilin-binding cyclosporins may also be used for example as a prophylactic treatment of neonates with congenital hepatic fibrosis or of transplant recipients, e.g. organ or tissue transplant recipients, e.g. liver transplant.

A cyclosporin is considered as binding to cyclophilin if it binds to human recombinant cyclophilin at least one fifth as well as does cyclosporin A in the competitive ELISA test described by Quesniaux in Eur. J. Immunol. 1987 17 1359-1365. In this test, the cyclosporin to be tested is added during the incubation of cyclophilin with coated BSA-cyclosporin A and the concentration required to give a 50% inhibition of the control reaction without competitor is calculated (IC₅₀). The results are expressed as the Binding Ratio (BR), which is the log to the base 10 of the ratio of the IC₅₀ of the test compound and the IC₅₀ in a simultaneous test of cyclosporin A itself. Thus a BR of 1.0 indicates that the test compound binds human cyclophilin one factor of ten less well than does cyclosporin A, and a negative value indicates binding stronger than that of cyclosporin A. The cyclosporins active against HCV have a BR lower than 0.7, preferably equal to or lower than zero.

Examples of non immunosuppressive cyclophilin-binding cyclosporins include e.g. compounds of Formula I

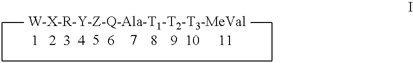

wherein
W is MeBmt, dihydro-MeBmt, 8′-hydroxy-MeBmt or O-acetyl-MeBmt¹;

X is □Abu, Val, Thr, Nva or 0-methyl threonine (MeOThr);

R is Pro, Sar, (D)-MeSer, (D)-MeAla, or (D)-MeSer(Oacetyl);
Y is MeLeu, thioMeLeu, y-hydroxy-MeLeu, Melle, MeVal, MeThr, MeAla, Mealle or MeaThr; N-ethylVal, N-ethyllle, N-ethylThr, N-ethylPhe, N-ethylTyr or N-ethylThr(Oacetyl);

Z is Val, Leu, MeVal or MeLeu;

Q is MeLeu, y-hydroxy-MeLeu, MeAla or Pro;

T₁ is (D)Ala or Lys;

T₂ is MeLeu or y-hydroxy-MeLeu; and

T₃ is MeLeu or MeAla.

Preferred compounds of formula I are e.g. compounds of formula Ia

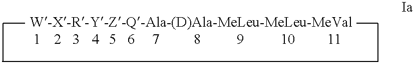

in which

W′ is MeBmt, dihydro-MeBmt or 8′-hydroxy-MeBmt;

X is □Abu, Val, Thr, Nva or 0-methyl threonine (MeOThr);

R′ is Sar, (D)-MeSer, (D)-MeAla, or (D)-MeSer(Oacetyl);
Y′ is MeLeu, y-hydroxy-MeLeu, Melle. MeVal, MeThr, MeAla, Mealle or MeaThr; N-ethylVal, N-ethyllle, N-ethylThr, N-ethylPhe, N-ethylTyr or N-ethylThr(Oacetyl);

Z is Val, Leu, MeVal or MeLeu; and

Q′ is MeLeu, y-hydroxy-MeLeu or MeAla.

The groups W′, X, Y′, Z. Q′ and R′ have, independently, the following preferred significances:

W′ is preferably W″ where W″ is MeBmt or dihydro-MeBmt;

X is preferably X′ where X′ is □Abu or Nva, more preferably X″ where X″ is □Abu;

R′ is preferably R″ where R″ is Sar;

Y′ is preferably Y″ where Y″ is y-hydroxy-MeLeu, MeVal, MeThr, Melle,

N-ethyllle or N-ethylVal;

Z is preferably Z′ where Z′ is Val or MeVal; and

Q′ is preferably Q″ where Q″ is MeLeu.

A preferred group of Compounds of formula Ia are those in which W′ is W″, X is X′, Y′ is Y″, Z is Z′, Q′ is Q″ and R′ is R″.

Examples of preferred compounds of Formula Ia are e.g.:

a) [dihydro-MeBmt]¹-[y-hydroxy-MeLeu]⁴-Ciclosporin; BR*=0.1; IR<1%

b) [MeVal]⁴-Ciclosporin; BR=0.1; IR<1%

c) [Melle]⁴-Ciclosporin; BR=−0.2; IR<1%

d) [MeThr]⁴-Ciclosporin

e) [y-hydroxy-MeLeu]⁴-Ciclosporin; BR=0.4; IR<1%

f) [Ethyl-lle]⁴-Ciclosporin; BR=0.1; IR<2%

g) [Ethyl-Val]⁴-Ciclosporin; BR=0; IR<2%

h) [Nva]²-[y-hydroxy-MeLeu]⁴-Ciclosporin;

i) [y-hydroxy-MeLeu]⁴- [y-hydroxy-MeLeu]⁶-Ciclosporin;

j) [MeVal]⁵-Ciclosporin; BR=0.4; IR=5.3%

k) [MeOThr]²-[(D)MeAla]³-[MeVal]⁵-Ciclosporin;

j) [8′-hydroxy-MeBmt]¹-Ciclosporin, BR=0.35; IR=1.8%

k) [MeAla]⁶-Ciclosporin; BR=−0.4; IR=3.2

l) [y-hydroxy-MeLeu]⁹-Ciclosporin; BR=0.15; IR=2.9

IR=Immunosuppressive Ratio, expressed as a percentage of the activity relative to Cyclosporin A.

Further examples of non-immunosuppressive cyclosporins are the compounds disclosed in WO 98/28330, WO 98/28329 and WO 98/28328, the contents thereof being incorporated herein by reference, e.g. compounds of formula II

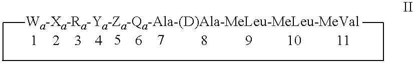

wherein Wₐ is

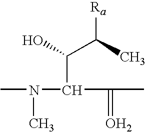

wherein Rₐ is a residue of formula 1c or 1d

—CH₂—CH═CH—CH₂—R₄Ic or —CH₂—SH—R′₄ Id

in which R₄ is C₁₋₄alkylthio, aminoC₁₋₄alkylthio, C₁₋₄alkylaminoC₁₋₄alkylthio, diC₁₋₄alkylamino-C₁₋₄alkylthio, pyrimidinylthio, thiazolylthio, N—C₁₋₄alkylimidazolylthio, hydroxyC₁₋₄alkylphenylthio, hydroxyC₁₋₄alkylphenoxy, nitrophenylamino or 2-oxopyrimidin1-yl, and R′₄ is C₁₋₄alkyl,

Xₐ is Abu;

Rₐ is —NMe—CH(R_{b})-CO-wherein R_{b} is H or —S-Alk-R₀ in which Alk-R₀ is methyl; or Alk is straight or branched C₂₋₆alkylene or C₃₋₆cycloalkylene and R₀ is H; OH; COOH; C₂₋₅alkoxy carbonyl; NR₁R₂ in which each of R₁ and R₂, independently, is selected from H. C₁₋₄alkyl, C₂₋₄alkenyl, C₃₋₆cycloalkyl and phenyl each optionally substituted by halogen, C₁₋₄alkoxy, C₂₋₅alkoxycarbonyl, amino, C₁₋₄alkylamino and/or diC₁₋₄alkyl-amino, and benzyl and a heterocyclic radical, said benzyl and heterocyclic radicals being saturated or unsaturated and containing 5 or 6 ring members and 1 to 3 heteroatoms, or R₁ and R₂ form, together with the nitrogen atom to which they are attached, a 4- to 6 membered heterocycle which may contain another heteroatom chosen from nitrogen, oxygen and sulphur, and which is optionally substituted by ₁₋₄alkyl, phenyl or benzyl; or each of R₁ and R₂, independently, is a radical of formula Ib

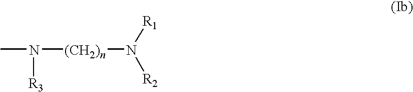

in which R₁ and R₂ are as defined above, R₃ is H or C₁₋₄alkyl and n is an integer ranging from 2 to 4;

Yₐ is MeLeu or y-hydroxy-MeLeu;

Zₐ is Val; and

Qₐ is MeLeu, with the proviso that R_{b} is not H when Yₐ is MeLeu, or a pharmaceutically acceptable salt thereof.

In the formula II, when R₁ and/or R₂ is a heterocyclic residue, it may be pyridyl, tetrahydro-pyridyl, piperidyl, imidazolyl, oxazolyl or thiazolyl. When R₁ and R₂ form a heterocyclic residue with the nitrogen atom to which they are attached, by way of example, the heterocyclic residue may be chosen from azetidinyl, piperidyl, piperazinyl, N-methyl-piperazinyl, N-phenylpiperazinyl, N-benzylpiperazinyl, pyridyl, imidazolyl, morpholino, thiomorpholino, tetrahydropyridyl, methyltetrahydropyridyl (for example 4-methyl-tetrahydropyridyl) or phenyltetrahydropyridyl (for example 4-phenyltetrahydropyridyl).

The Compounds of formula I, Ia or II may be obtained in a variety of ways, which may be classified as:

1) Fermentation

2) Biotransformation

3) Derivatisation

4) Partial Synthesis

5) Total Synthesis
as disclosed e.g. in EP 0 484 281 A1, WO 00/01715, WO 98/28330, WO 98/28329 or WO 98/28328 the contents thereof being incorporated herein by reference.

In a series of further specific or alternative embodiments, the present invention also provides:

1.1 A method for preventing or treating conditions associated with liver diseases in a subject in need thereof, comprising administering to said subject a therapeutically effective amount of a non-immunosuppressive cyclophilin-binding cyclosporin, e.g. a compound of formula I, Ia or II.

According to the invention, the non-immunosuppressive cyclophilin-binding cyclosporin may be administered in an amount effective to alleviate or eliminate one or more of the signs, symptoms or conditions associated with liver diseases, for example, effective to suppress the production of collagen measured in a biopsy sample of a subject.

1.2 A method for suppressing HSC growth in a medium, comprising applying to this medium an effective amount of a non-immunosuppressive cyclophilin-binding cyclosporin, e.g. a compound of formula I, Ia or II.

1.3 A method for inhibiting conditions associated with liver disease in a patient in need thereof, comprising administering to the subject a therapeutically effective amount of a non-immunosuppressive cyclophilin-binding cyclosporin, e.g. a compound of formula I, Ia or II.

1.4 A method for preventing or treating the recurrence of conditions associated with liver diseases in a transplant recipient in need thereof, comprising administering to said recipient a therapeutically effective amount of a non-immunosuppressive cyclophilin-binding cyclosporin, e.g. a compound of formula I, Ia or II.

2. Use of a non-immunosuppressive cyclophilin-binding cyclosporin, e.g. a compound of formula I, Ia or II, in the preparation of a pharmaceutical composition for use in any method as defined above.

3. A pharmaceutical composition for use in any method as defined above, comprising a non-immunosuppressive cyclophilin-binding cyclosporin, e.g. a compound of formula I, Ia or II, together with one or more pharmaceutically acceptable diluents or carriers therefore.

Utility of the non-immunosuppressive cyclophilin-binding cyclosporins (hereinafter “cyclosporins of the invention”) in treating diseases and conditions as hereinabove specified may be demonstrated in standard animal or clinical tests, e.g. in accordance with the methods described hereinafter.

A. In vitro Cell culture: HSCs are isolated from the liver of male Wistar rats by sequential in situ perfusion with collagenase and digestion with pronase, followed by centrifugation in a double-layered (17%/11.5%) metrizamide solution (Sigma. Chemical, St. Louis, Mo.), as described in Kato, et al, J. Hepatol (1999) 31:91-99. HSCs are cultured in Dulbecco's modified Eagle's medium (DMEM) with 10% fetal calf serum (FCS). Experiments are performed on cells between the third and fourth serial passages. To measure mouse matrix metalloproteinase (MMP-1) and tissue inhibitor of matrix metalloproteinase (TIMP-1), TWNT-4 cells which is a human cell line derived from HSCs as produced according to Shibata, et al, Cell Transplant (2003) 12:499-507 is used for evaluating the effects of Fasudil on MMP-1 and TIMP-1. TWNT-4 cells are cultured in DMEM with 10% FCS as reported previously (Id). NIM811 (Novartis Pharma AG, Basel, Switzerland) is dissolved in DMEM and added to cultures. Cell viability of HSCs is more than 90% under serum-free conditions for 24 h in the presence of 2 μM NIM811.

Type 1 collagen assay: Cultured HSCs are incubated in serum-free medium in the presence or absence of NIM811 for 24 h. Type I collagen is determined in culture media by ELISA as described in Iwamoto, et al, J. Hepatol (2000) 32:762-770. Anti-rat type I collagen antibody (LSL, Tokyo, Japan) may be used as the primary antibody. Peroxidase-conjugated goat-anti-rabbit IgG (Organon Teknika Corporation, Durham, N.C.) is used as the secondary antibody. Rat tail tendon collagen type I (Advance Biofactures Corporation, Lymbrook, N.Y.) is used as a standard control.

MMP-1, TIMP-1, and collagenase assay: Cultured TWNT-4 cells are incubated in serum-free medium in the presence or absence of NIM811 for 24 h. MMP-1 and TIMP-1 production are determined in culture media by ELISA with a Biotrak ELISA system for human MMP-1 (Amersham Biosciences, Piscataway, N.J., USA) and an hTIMP-1 kit (Daiichi Fine Chemical Co. Ltd., Toyama, Japan), respectively following procedures described in Fukushima, et al, Liver Int (2005) 25:829-838. Active MMP-1 and pro-MMP-1 in culture media are determined with a MMP-1 Biotrak Activity Assay System (Amersham).

Analysis of gene expression using real time RT-PCR: Total RNA is prepared from TWNT-4 cells with Trizol reagent (Invitrogen, Carlsbad, Calif., USA), which are maintained in either the presence or absence of NIM811 in 10% FCS for 24 h. cDNA is synthesized from 1.0 mg RNA with GeneAmp™ RNA PCR (Applied Biosystems, Branchburg, N.J., USA) using random hexamers. Real-time PCR is performed using LightCycler-FastStart DNA Master SYBR Green 1 (Roche, Tokyo, Japan) as described in Nakamuta, et al, Int J. Mol Med (2005) 16(4):631-635. A reaction mixture (20 μl) is used containing LightCycler-FastStart DNA Master SYBR Green 1, 4 mM MgCl₂, 0.5 μM of the upstream and downstream PCR primers, and 2 ml of the first strand cDNA as a template. To control for variations in the reactions, all PCRs are normalized against glyceraldehyde-3-phosphate dehydrogenase (GAPDH) expression. The primers which may be used are as follows: 5′-AGGGTGAGACAGGCGAACAG-3′ (forward primer) (SEQ ID NO. 1) and 5′-CTCTTGAGGTGGCTGGGGCA-3′ (reverse primer)(SEQ ID NO. 2) for human type I collagen a1 chain (GenBank™ accession number NM-000088) (21); 5′-AATGAGATGGCCACTGCCGC-3′ (forward primer)(SEQ ID NO. 3) and 5′-CAGAGTATTTGCGCTCCGGA-3′ (reverse primer) for human a-SMA (GenBank™ accession number NM-000088); 5′-GATCATCGGGACAACTCTCCT-3′ (forward primer) and 5′-TCCGGGTAGAAGGGATTTGTG-3′ (reverse primer) for MMP-1 (GenBank™ accession number NM002421); 5′-TTCTGCAATTCCGACCTCGT-3′ (forward primer) and 5′-TCCGTCCACAAGCAATGAGT-3′ (reverse primer)(SEQ ID NO. 4) for TIMP-1 (GenBank™ accession number NM003254); 5′-GGATCTCAGGCATTCCTCGG-3′ (forward primer)(SEQ ID NO. 5) and 5′-CAGTATGCCACCACGCACCA-3′ (reverse primer)(SEQ ID NO. 6) for Smad7 (Quan, et al, J Biol Chem (2005) 80:8079-8085); 5′-GGCCGTTTGTATGTGCACCCTC-3′ (forward primer)(SEQ ID NO. 7) and 5′-GGGCGATCTAATGAAGGGTCC-3′ (reverse primer)(SEQ ID NO. 8) for transforming growth factor □ receptor I (TGFβRI) (Woszcyk et al, Med Sci Monit (2004) 10:C33-C37)).

Analysis of BrdU incorporation: HSC incorporation of BrdU is measured using a cell proliferation ELISA (Roche Diagnostics GmbH, Mannheim, Germany) as described in Higashi, et al, J. Lab Clin Med (2005) 145(6):316-322. Briefly, subconfluent HSCs are serum starved for 24 h. They are then washed with DMEM and incubated for 24 h with BrdU in DMEM with 10% FCS in the presence or absence of NIM811. After labeling the cells with BrdU, cellular DNA is digested and incubated with the anti-BrdU antibody conjugated with peroxidase. BrdU incorporation is estimated by measuring the fluorescence intensity of the supernatant at 450 nm (excitation) and 690 nm (emission).

Analysis of Apoptosis: HSCs are maintained in either the presence or absence of NIM811 in serum-free conditions for 24 h. Cells are fixed for 30 min in 4% paraformaldehyde/PBS at room temperature, and permeabilized for 5 min in PBS containing 0.2% Triton X-100 at 4° C. Cells are then stained with Hoechst 33342, and analyzed by the TUNEL method using an In Situ Cell Death Detection Kit (Roche) according to the manufacturer's instructions. The samples are visualized with an LSM 510 confocal laser scanning microscope (Zeiss). At least 100 cells from three independent experiments and from three different cell preparations are counted for each condition.

Western blot analysis for phospho- and nonphospho-MAPKs: Western blot analysis is performed as described in Uchimura, et al, Hepatology (2001) 33:91099. HSCs are starved for 24 h, then treated with or without NIM811 for 2 h. Whole cell lysates containing 1×10⁷ TWNT-4 cells are prepared in 100 μl SDS-PAGE sample buffer. Protein lysates are subjected to 12% SDS-PAGE, transferred to a polyvinylidene difluoride membrane (Millipore, Bedford, Mass.), and probed with the primary antibody for ERK1/2 MAPK, phospho-ERK1/2 MAPK (Thr202/Tyr204), JNK, phospho-JNK (Thr183/Tyr185), p38 MAPK, or phospho-p38 MAPK (Thr180/Tyr182) (New England Biolabs, Beverly, Mass.). Antibody binding is detected using peroxidase linked anti-rabbit IgG (Amersham Pharmacia Biotech, Piscataway, N.J.) as the secondary antibody. The blots are developed using ECL-plus (Amersham Pharmacia Biotech, Piscataway, N.J.) to visualize the antibodies. The levels of ERK1/2 MAPK, phosphorylated-ERK1/2 MAPK, JNK, phosphorylated-JNK, p38 MAPK, and phosphorylated-p38 MAPK are quantitated by densitometry using an optical scanner system. For comparison, the ratios of phosphorylated ERK1/2, JNK, and p38 MAPK to nonphosphorylated ERK1/2, JNK, and p38 MAPK, respectively, are calculated from the densitometric data.

Western blot analysis for phospho- and nonphospho-Smad2 and Smad3: Western blot analysis is performed as described above for MAPK analysis and probed with a primary antibody for Smad2, phospho-Smad2 (Thr/Tyr), Smad3, or phospho-Smad3 (Thr/Tyr) (Cell Signaling Technology, Danvers, Mass.). For comparison, the ratios of phosphorylated Smad2 and Smad3 to nonphosphorylated Smad2 and Smad3, respectively, are calculated from densitometric data.

Statistical Analysis: All results are shown as the mean ± SEM. Comparisons are made using one-way ANOVA followed by Scheffe's test or the Mann-Whitney test.

### EXAMPLE 1

### Effects of NIM811 on Type I Collagen Accumulation, MMP-1 and TIMP-1 Production, and Collagenase Activity

To assess the effect of NIM811 on ECM production by HSCs, is determined type I collagen concentrations in culture media after adjusting the number of rat HSCs as described above. Treatment of the cells with increasing concentrations of NIM811 as well as cyclosporine leads to a concentration-dependent suppression of collagen accumulation; 0.5 μM NIM811 reduces collagen accumulation by approximately 50%. This suppression is regulated at least as far upstream as the transcriptional level because treatment with NIM811 suppresses collagen expression. As previously reported in Nakamuta, et al, Transplant Proc (2005) 37:4598-4602, cyclosporin at a clinically relevant concentrations of 0.125 μM (150 ng/ml) reduces collagen concentration by approximately 50%, whereas tacrolimus at a clinically relevant concentration of 12.5 nM (10 ng/ml) does not significantly reduces collagen production. Collagen accumulation, in addition to being determined by the rate of collagen production, is regulated by collagenase activity, namely, by the balance between MMP-1 and TMIP-1. NIM811 leads to a concentration-dependent increase in collagenase activity (active MMP-1) and pro-MMP-1 levels; in the presence of 0.5 μM NIM811, collagenase activity increased roughly 2-fold. However, NIM811 even at concentration of 2.0 μM does not significantly reduce TIMP-1 production.

### EXAMPLE 2

### Effects of NIM811 on Type 1 Collagen, MMP-1, and TIMP-1 Gene Expression

RT-PCR is performed as described herein to evaluate the effects of NIM811 or cyclosporin on the mRNA levels of type I collagen, MMP-1, and TIMP-1. The expression of type I collagen is reduced in the presence of 0.5 μM NIM811 by roughly 30%. In contrast, 0.5 μM NIM811 increases the expression of MMP-1 nearly 2-fold but it did not change that of TIMP-1. These results indicate the effects of NIM811 on gene expression are similar to its effect on protein production.

### EXAMPLE 3

### Effect of Fasudil on Cell Proliferation and Apoptosis

Previous work has demonstrated that, in addition to stimulating collagen production, activated HSCs inhibits the degradation of interstitial collagens by interstitial collagenases such as MMP-1, indicating that matrix degradation is inhibited during the progression of fibrosis (see Benyon, et al, Gastroenterology (1996) 110:821-831, Iredale, et al, Hepatology (1996) 24:17-184, Iredale, et al, J. Clin Invest (1992) 90:282-287). TIMP-1 has been reported to regulate cell growth and apoptosis independently of the inhibition of matrix degradation (see Murphy, et al, J. Biol Chem (2002) 277:11069-11076). NIM811 suppresses cell growth of HSCs in a concentration-dependent manner without apoptosis.

BrdU incorporation is measured as described herein to investigate the effect of NIM811 on cell proliferation. Quantitative analysis shows that 2.0 μM NIM811 treatment decreases new DNA synthesis by nearly 30% although less concentration treatment did not. Further, in the presence of 2 μM NIM811, no apoptosis is observed. Our results indicate that NIM has therapeutic potential for liver fibrosis through suppression of collagen production and enhancement of collagenase activity.

### EXAMPLE 4

### Effects of NIM811 on MAPKs Signaling Pathways

To explore a mechanism by which NIM811 suppresses collagen production and cell proliferation and enhances collagenase activity, the effects of NIM811 on intra-cellular signaling cascades, such as MAPKs including ERK1/2, JNK, and p38 which play important roles in collagen production and cell proliferation in HSCs (Marr, et al, Hepatology (2000) 1:428-434) is assessed as described above. NIM811 at a concentration of 0.5 μM enhances phosphorylation of JNK and p38 MAPK by nearly 3.6-fold and 2.3-fold, respectively. Treatment with NIM811 significantly enhances phosphorylation of JNK and p38 MAPK in a concentration manner, but does not suppress ERK1/2. It has been previously demonstrated that cyclosporine exerts its immunosuppressive effects through both the calcineurin-dependent NFAT pathway and calcineurin-independent activation pathway for JNK and p38 (Mastuda, et al, EMPO Reports (2000) 1:428-434)). NIM811, an analogue of cyclosporine, does not activate NFAT pathway because it does not bind to cyclophilin A (Waldmeier, et al, Mol Pharmacol, (2002) 62(1):22-29). The different effects on JNK and p38 between NIM811 and cyclosporine might be derived from the absence of NFAT pathway in NIM811.

### EXAMPLE 5

### Effects of NIM811 on TGFβ Signaling Pathways

In addition to MAPKs, TGF-β signaling cascades strongly stimulate collagen production HSCs (Friedman, et al, J Biol Chem (1994) 269:10551-10558). TGF-β binds to TGFβRII on the cell membrane, and then TGFβRII phosphorylates TGFβRI at the serine and threonine residues located glycine/serine-rich domain (Wrana, et al, Cytokine Growth Factor Rev (2000) 11:5-13). The phosporylated TGFβRI phosphorylates Smad2 and Smad3 at a C-terminal SSXS motif, which form a complex with common partner Smad4. These Smad proteins translocate to the nucleus and activate the transcription of target genes such as collagen (Id). Since TGFβ signal cascades through Smad2 and Smad3 strongly regulate the expression of type I collagen gene as described in Friedman, et al, J Biol Chem (1994) 269:10551-10558, the effects of NIM811 on phosphorylation of Smad2 and Smad3 is evaluated. Treatment with NIM811 significantly suppresses phosphorylation of Smad2 and Smad3 in a concentration manner; 0.5 μM NIM811 suppresses phosphorylation of Smad2 and Smad3 by nearly 70% and 60%, respectively. The expression of Smad7 negatively regulates TGFβ signaling pathways by inhibition of TGFβRII phosphorylation (Hayashi, et al, Cell (1997) 1165-1173). 0.5 μM NIM 811 enhances expression of Smad7 nearly 2-fold, and suppresses TGFβRI by nearly 50%. These results suggest that NIM811 may inhibit the kinase activity TGFβRII and/or TGFβR1. Smad7 (Id), immunophilin FKBP (FK506-binding protein) 12 (40), and SARA (Smad anchor for receptor activation) (41), associate with TGFβR and regulate TGFβ signaling. NIM811 enhances expression of Smad7 and suppresses that of TGF□RI, indicating that NIM811 inhibits TGFβ signaling pathways at least partially through blockade at receptor level. Cyclosporine also has similar effects on Smad2, Smad3, Smad7, and TGFβRI (unpublished data).

As described before, NIM811 had the opposite effects on JNK and p38 to cyclosporine although both of them showed similar effects on collagen production and cell proliferation, suggesting that NIM and cyclosporine exhibit anti-fibrogenic effects mainly blockade of TGFβ signaling pathways.

Cyclophilins are a family of PPlases, which catalyze the cis-trans interconversion of peptide bounds amino-terminal to proline residues, facilitating changes in protein conformation (Waldmeier, supra). There are more than ten subtypes of cyclophilin, and they are involved in numerous cellular processes, including transcriptional regulation, immune response, protein secretion and mitochondrial function (Waldmeier supra, Duina, et al, Science (1996) 274:1713-1715). Watashi et al., Hepatology (2003) 38:1282-1288, recently reported that NIM811 suppresses replication of HCV replication in vitro, whereas tacrolimus did not show this effect. Since NIM811 lacks the ability to bind to cyclophilin A (14), NIM811 appears to exert its pharmacological effects by binding to other cyclophilin, such as cyclophilin B or D. Notably, NIM811 shows antiviral effects via binding cyclophilin B which is a functional regulator of HCV RNA polymerase (Watashi, et al, Mol Cell (2005) 19:111-122). NIM811 also has been reported to have cytoprotective properties depending on interference of the interaction with cyclophilin D which regulates the mitochondrial permeability transition (Waldmeier, et al, Mol Pharmacol (2002) 62:22-29). Kon et al, Hepatology (2004) 40:1170-1179 reported that NIM811 prevented acetaminophen-induced necrosis and apoptosis of cultured mouse hepatocytes.

B. Clinical Trial

A total of 15 patients with hepatic fibrosis/cirrhosis are enrolled in a study of 2 weeks. Each patient receives a cyclosporine of the invention, e.g. [Melle]4-ciclosporin, at a dose of 7 to 15 mg/kg p.o. The serum levels of Hepatitis C antigens are determined at day 0 and day 14 in each patient.

A person suffering from hepatic fibrosis/cirrhosis, in particular liver damage, may exhibit one or more of the following signs or symptoms: (a) elevated ALT, (b) positive test for anti-HCV antibodies, (c) presence of HCV as demonstrated by a positive test for HCV-RNA, (d) clinical stigmata of chronic liver disease, (e) hepatocellular damage. Such criteria may not only be used to diagnose Hepatitis hepatic fibrosis/cirrhosis, but can be used to evaluate a patient's response to drug treatment.

Elevated serum alanine aminotransferase (ALT) and aspartate aminotransferase (AST) are known to occur in uncontrolled Hepatitis C, and a complete response to treatment is generally defined as the normalization of these serum enzymes, particularly ALT (Davis et al., 1989, New Eng. J. Med. 321:1501-1506). ALT is an enzyme released when liver ceils are destroyed and is symptomatic of HCV infection.

In order to follow the course of HCV replication in subjects in response to drug treatment, HCV RNA may be measured in serum samples by, for example, a nested polymerase chain reaction assay that uses two sets of primers derived from the N53 and N54 non-structural gene regions of the HCV genöme. Farci et al., 1991, New Eng. J. Med. 325:98-104. Ulrich et al., 1990, J. Clin. Invest., 86:1609-1614.

Histological examination of liver biopsy samples may be used as a second criteria for evaluation. See, e.g., Knodell et al., 1981, Hepatology 1:431-435, whose Histological Activity Index (portal inflammation, piecemeal or bridging necrosis, lobular injury and fibrosis) provides a scoring method for disease activity.

Daily dosages required in practicing the method of the present invention will vary depending upon, for example, the non-immunosuppressive cyclophilin-binding cyclosporin employed, the host, the mode of administration, the severity of the condition to be treated. A preferred daily dosage range is about from 1 to 50 mg/kg per day as a single dose or in divided doses.

Suitable daily dosages for patients are on the order of from e.g. 1 to 20 mg/kg p.o or i.v. Suitable unit dosage forms for oral administration comprise from ca. 0.25 to 10 mg/kg active ingredient, e.g. [Melle]4-ciclosporin, together with one or more pharmaceutically acceptable diluents or carriers therefor.

The cyclosporins of the invention may be administered by any conventional route, in particular enterally, e.g. orally, for example in the form of solutions for drinking, tablets or capsules or parenterally, for example in the form of injectable solutions or suspensions. Preferred pharmaceutical compositions may be e.g. those based on microemulsions as described in UK 2,222,770 A.

The cyclosporins of the invention may be administered as the sole ingredient or together with other drugs, e.g. a drug which has anti-HCV activities, e.g. an interferon, e.g. interferon-∝2a or interferon-□-2b, e.g. Intron^{R} A, Roferon^{R}, Avonex^{R}, Rebif^{R} or Betaferon^{R}, or an interferon conjugated to a water soluble polymer or to human albumin, e.g. albuferon, an anti-viral agent, e.g. ribovirin, lamivudine, NV08 or NM283, an inhibitor of the HCV encoded factors like the NS3/4A protease, the helicase or RNA polymerase or a prodrug of such an inhibitor, an anti-fibrotic agent, e.g. a N-phenyl-2-pyrimidine-amine derivative, e.g. imatinib, an immune modulating agent, e.g. mycophenolic acid, a salt or a prodrug thereof, e.g. sodium mycophenolate or mycophenolate mofetil, or a S1 P receptor agonist, e.g. FTY720 or an analogue thereof optionally phosphorylated, e.g. as disclosed in EP627406A1, EP778263A1, EP1002792A1, WO02/18395, W002/76995, WO 02/06268, JP2002316985, WO03/29184, W003/29205, W003/62252 and W003/62248.

Conjugates of interferon to a water-soluble polymer are meant to include especially conjugates to polyalkylene oxide homopolymers such as polyethylene glycol (PEG) or polypropylene glycols, polyoxyethylenated polyolis, copolymers thereof and block copolymers thereof. As an alternative to polyalkylene oxide-based polymers, effectively non-antigenic materials such as dextran, polyvinyl pyrrolidones, polyacrylamides, polyvinyl alcohols, carbohydrate-based polymers and the like can be used. Such interferon-polymer conjugates are described in U.S. Pat. Nos. 4,766,106, 4,917,888, European Patent Application No. 0 236987, European Patent Application No. 0 510 356 and international Application Publication No. WO 95/13090. Since the polymeric modification sufficiently reduces antigenic responses, the foreign interferon need not be completely autologous. Interferon used to prepare polymer conjugates may be prepared from a mammalian extract, such as human, ruminant or bovine interferon, or recombinantly produced. Preferred are conjugates of interferon to polyethylene glycol, also known as pegylated interferons.

Especially preferred conjugates of interferon are pegylated alfa-interferons, for example pegylated interferon-a-2a, pegylated interferon-a-2b; pegylated consensus interferon or pegylated purified interferon-a product. Pegylated interferon-□-2a is described e.g. in European Patent 593868 and commercially available e. g. under the tradename PEGASUS (Hoffmann-La Roche). Pegylated interferon-a-2b is described, e.g. in European Patent 975,369 and commercially available e.g. under the tradename PEG-INTRON An (Schering Plough). Pegylated consensus interferon is described in WO 96/11953. The preferred pegylated ∝-interferons are pegylated interferon-∝2a and pegylated interferon-∝-2b. Also preferred is pegylated consensus interferon.

Daily dosages with respect to the co-agent used will vary depending upon, for example, the compound employed, the host, the mode of administration and the severity of the condition to be treated. For example, lamivudine may be administered at a daily dosage of 100 mg.

The pegylated interferon may be administered parenterally one to three times per week, preferably once a week, at a total weekly dose ranging from 2 to 10 million IU, more preferable 5 to 10 million IU, most preferable 8 to 10 million IU.

In accordance with the foregoing the present invention provides in a yet further aspect:

4. A pharmaceutical combination comprising a) a first agent which is a non-immunosuppressive cyclophilin-binding cyclosporin, e.g. a compound of formula 1, Ia or il. and b) a co-agent, e.g. a second drug agent as defined above, e.g. for use in any method as defined above.

5. A method as defined above comprising co-administration, e.g. concomitantly or in sequence, of a therapeutically effective amount of a non-immunosuppressive cyclophilin-binding cyclosporin, e.g. a compound of formula 1, Ia or 11, and a co-agent, e.g. a second drug agent as defined above.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The administration of a pharmaceutical combination of the invention results in a beneficial effect, e.g. a synergistic therapeutic effect, compared to a monotherapy applying only one of its pharmaceutically active ingredients. A preferred synergistic combination is a combination of a non-immunosuppressive cyclophilin-binding cyclosporin with an interferon, optionally conjugated to a polymer.

A further preferred combination is a combination of a non-immunosuppressive cyclophilin- binding cyclosporin with mycophenolic acid, a salt or a prodrug thereof, or with a S1 P receptor agonist, e.g. FTY720.

[Melle]4-ciclosporin or [MeVal]4-Ciclosporin is a preferred non-immunosuppressive cyclophilin-binding cyclosporin for use according to the invention.

1. Use of a cyclosporin in the preparation of a pharmaceutical composition for preventing or treating conditions associated with liver diseases wherein the closporin (i) binds to human recombinant cyclophilin with a binding ratio (BR) of less than 0.7, BR being the log to the base 10 of the ratio of the IC50 of the cyclosporin to the IC50 in a simultaneous test of cyclosporin A as measured in a competitive ELISA test; and (ii) has an activity in the Mixed lymphocyte Reaction of not more than 5% that of cyclosporin A.

2. Use of a cyclosporin according to claim 1 in the preparation of a pharmaceutical composition for inhibiting liver diseases.

3. Use of a cyclosporin according to claim 1 in the preparation of a pharmaceutical composition for preventing the recurrence of liver diseases in a transplant recipient.

4. Use according to claim 1, wherein the cyclosporin is a compound of Formula I wherein
W is MeBmt, dihydro-MeBmt, 8′-hydroxy-MeBmt or O-acetyl-MeBmt4;
X is ocAbu, Val, Thr, Nva or 0-methyl threonine (MeOThr); R is Pro, Sar, (D) -MeSer, (D)-MeAla, or (D)-MeSer(Oacetyl); Y is MeLeu, thioMeLeu,
y-hydroxy-MeLeu, Melle, MeVal, MeThr, MeAla, Mealle or MeaThr; N-ethylVal,
N-ethylile, N-ethylThr, N-ethylPhe, N-ethylTyr or N-ethylThr(Oacetyl) Z is Val, Leu, MeVal or MeLeu, Q is MeLeu, y-hydroxy-MeLeu, MeAla or Pro, T′ is (D)Ala or Lys, T2 is MeLeu or y- hydroxy-MeLeu, and T3 is MeLeu or MeAla; a compound of Formula Ia-15 W′-X R′ Y′ Z-Q′-Ala-(D)Ala-MeLeu-MeLeu-MeVal-1 2 3 4 5 6 7 8 9 10 11 Ia in which W′ is MeBmt, diNydro-MeBmt or 8′-hydroxy-MeBmt; X is ocAbu, Val, Thr, Nva or 0-methyl threonine (MeOThr); R′ iS Sar, (D)-MeSer, (D)-MeAla, or (D)-MeSer(Oacetyl); Y′ is MeLeu, y-hydroxy-MeLeu, Melle, MeVal, MeThr, MeAla, Mealle or MeaThr; N-ethylVal, N-ethylile, N-ethylThr, N-ethylPhe,
N-ethylTyr or N-ethylThr(Oacetyl) Z is Val, Leu, MeVal or MeLeu; and Q′ is MeLeu, y-hydroxy-MeLeu or MeAia. Or a compound of formula 11 WXa Ra Ya Za Qa Ala-(D)Ala-MeLeu-MeLeu-MeVal-1 2 3 4 5 6 7 8 9 10 11 11 wherein Wa is Ra HO/ . . . CH CH I I CH3 OH2 wherein Ra is a residue of formula Ic or Id
CH2—CH—CH—CH2 R4 Ic or CH2 SI I R′4 Id in which R4 IS C′4alkylthio, aminoC 4alkylthio, C 4alkylaminoC′4alkylthio, diC′4alkylamino-C 4alkylthio, pyrimidinylthio, thiazolylthio, N—C 4alkylimidazolylthio, hydroxyC, 4alkylphenylthio, hydroxyC′4alkylphenoxy, nitrophenylamino or 2-oxopyrimidin-1-yl, and R′4 is C′ 4alkyl, Xa is Abu; −16 Ra is —NMe—CH(Rb)—CO— wherein Rb is H or —S— Alk-R0 in which Alk-Ro is methyl; or
Alk is straight or branched C2 6alkylene or Cal cycloalkylene and Ro is H;
OH; COOH; C2 5alkoxy-carbonyl; NRR2 in which each of R. and R2, independently, is selected from H. C′-alkyl, C2 alkenyl, C3 6cycloalkyl and phenyl each optionally substituted by halogen, C, alkoxy, C25alkoxycarbonyl, amino, C-alkylamino and/or diC′-alkyl-amino, and benzyl and a heterocyclic radical, said benzyl and heterocyclic radicals being saturated or unsaturated and containing 5 or 6 ring members and 1 to 3 heteroatoms, or R. and R2 form, together with the nitrogen atom to which they are attached, a 4- to 6 membered heterocycle which may contain another heteroatom chosen from nitrogen, oxygen and sulphur, and which is optionally substituted by C-alkyl, phenyl or benzyl; or each of R′ and R2, independently, is a radical of formula lb i:′) /.t (in which R and R2 are as defined above, R3 is H or C′ alkyl and n is an integer ranging from 2 to 4; Ya is MeLeu or y-hydroxy-MeLeu; Za is Val; and Qa is MeLeu, with the proviso that Rb is not H when Ya is MeLeu, or a pharmaceutically acceptable salt thereof.

5. A pharmaceutical composition for preventing or treating liver diseases comprising a cyclosporin according to claim 1 together with one or more pharmaceutically acceptable diluents or carriers therefor.

6. A pharmaceutical combination comprising a) a first agent which is a cyclosporin according to claim 1, and b) a co-agent having anti-fibrogenic properties.

7. A pharmaceutical combination for use in the prevention or treatment of Cirrhosis, comprising a) a first agent which is a cyclosporin according to claim 1, and b) a co-agent selected from an agent having anti-HCV properties, an anti-fibrotic agent, an immune modulating agent or a S1 P receptor agonist—17.

8. A method for preventing or treating liver diseases in a subject in need thereof,
comprising administering to said subject a therapeutically effective amount of a cyclosporin according to claim 1.

9. A method for suppressing HSC growth in a medium, comprising applying to this medium an effective amount of a cyclosporin according to claim 1.

10. A method for inhibiting liver diseases in a patient in need thereof, comprising administering to this subject a therapeutically effective amount of a cyclosporin according to claim 1.

11. A method for preventing the recurrence of liver diseases in a transplant recipient in need thereof, comprising administering to said recipient a therapeutically effective amount of a cyclosporin according to claim 1.

12. A method according to any claim 8, comprising co-administration concomitantly or in sequence of a therapeutically effective amount of a cyclosporin as defined in claim 1 and a co-agent selected from an agent having anti-HCV properties, an anti-fibrotic agent, an immune modulating agent or a SIP receptor agonist.
